(19) United States
(12) Patent Application Publication
(10) Pub. No.: US 2015/0105764 A1
(43) Pub. Date: Apr. 16, 2015

(54) COMPLIANT CRYOBALLOON APPARATUS FOR DENERVATION OSTIA OF THE RENAL ARTERIES

(71) Applicant: BOSTON SCIENTIFIC SCIMED, INC., MAPLE GROVE, MN (US)

(72) Inventors: RAED N. RIZQ, MAPLE GROVE, MN (US); ROGER N. HASTINGS, MAPLE GROVE, MN (US); DAVID J. SOGARD, EDINA, MN (US)

(73) Assignee: BOSTON SCIENTIFIC SCIMED, INC., MAPLE GROVE, MN (US)

(21) Appl. No.: 14/572,042

(22) Filed: Dec. 16, 2014

Related U.S. Application Data
(63) Continuation of application No. 12/980,952, filed on Dec. 29, 2010.
(60) Provisional application No. 61/291,476, filed on Dec. 31, 2009.

(51) Int. Cl. A61B 18/02

(52) U.S. Cl. CPC A61B 18/02; A61B 2018/0022
USPC 606/23

(57) ABSTRACT

A cryotherapy balloon catheter includes a compliant cryotherapy balloon comprising a distal balloon section dimensioned for placement within a renal artery and a proximal balloon section dimensioned to abut against an ostium of the renal artery and extend into at least a portion of the abdominal aorta. The compliant balloon has a diameter that varies non-uniformly along a length of the compliant balloon, such that a diameter at the proximal balloon section is larger than a diameter of the distal balloon section. The cryotherapy balloon catheter may be configured to deliver cryogenic therapy to at least the ostium of the renal artery sufficient to irreversibly terminate renal sympathetic nerve activity, such as by causing neurotmesis of renal nerve fibers and ganglia at the ostium of the renal artery.

### RELATED PATENT DOCUMENTS

This application is a continuation of U.S. patent application Ser. No. 12/980,952, filed Dec. 29, 2010; which claims the benefit of Provisional Patent Application Ser. No. 61/291,476 filed on Dec. 31, 2009, to which priority is claimed under 35 U.S.C. §119(e), and which are incorporated herein by reference.

### TECHNICAL FIELD

The present invention is related to systems and methods for improving cardiac and/or renal function through neuromodulation, including disruption and termination of renal sympathetic nerve activity.

### BACKGROUND

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

### SUMMARY

Devices, systems, and methods of the present invention are directed to modifying renal sympathetic nerve activity using cryotherapy. Embodiments of the present invention are directed to a cryotherapy balloon catheter apparatus that includes a flexible shaft comprising a proximal end, a distal end, and a lumen arrangement extending between the proximal and distal ends. A compliant balloon is provided at the distal end of the shaft and fluidly coupled to the lumen arrangement. The compliant balloon is arranged generally lengthwise along a longitudinal section of the distal end of the shaft and adapted to inflate in response to receiving pressurized cryogenic fluid and to deflate in response to removal of the cryogenic fluid. A hinge mechanism is provided on the flexible shaft proximal of the compliant balloon. The hinge mechanism is configured to facilitate preferential bending at the distal end of the shaft to aid in directing the compliant balloon into the renal artery from the abdominal aorta.

A compliant cryotherapy balloon of the present invention preferably comprises a distal balloon section dimensioned for placement within a renal artery and a proximal balloon section dimensioned to abut against an ostium of the renal artery and extend into at least a portion of the abdominal aorta. The compliant balloon preferably has a diameter that varies non-uniformly along a length of the compliant balloon, such that a diameter at the proximal balloon section is larger than a diameter of the distal balloon section.

Embodiments of a cryotherapy balloon catheter apparatus of the present invention may be configured to deliver cryogenic therapy to at least the ostium of the renal artery sufficient to terminate renal sympathetic nerve activity along at least the renal artery ostium. Embodiments of a cryotherapy balloon catheter apparatus may be configured to deliver cryogenic therapy to at least the ostium of the renal artery sufficient to cause neurotmesis of renal nerve fibers and ganglia at the ostium.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery, which includes the ostium of the renal artery;

FIGS. 3B and 3C illustrate a portion of a renal nerve;

FIG. 4 illustrates a cryotherapy balloon catheter deployed at the ostium of a renal artery in accordance with embodiments of the present invention;

FIG. 5A illustrates the distal portion of a cryoballoon catheter configured for deployment at the ostium, and within the lumen, of a renal artery in accordance with embodiments of the present invention;

FIG. 5B illustrates the distal portion of a cryoballoon catheter configured for deployment at the ostium, and within the lumen, of a renal artery in accordance with other embodiments of the present invention;

FIGS. 5C and 5D illustrate embodiments of a patterned cryotherapy arterial section of a cryoballoon in accordance with embodiments of the present invention;

FIGS. 5E and 5F illustrate embodiments of a patterned cryotherapy arterial section of a cryoballoon comprising dual balloon sections in accordance with other embodiments of the present invention;

FIGS. 6-8 are cross-sections of a cryoballoon in accordance with various embodiments of the present invention;

FIGS. 9-11 are different views of a cryoballoon catheter implemented in accordance with embodiments of the present invention;

FIG. 12 illustrates a portion of the cryoballoon catheter that incorporates a hinge mechanism in accordance with embodiments of the present invention;

FIGS. 13-16 illustrate a series of views of a cryoballoon catheter at different states of deployment within a patient in accordance with embodiments of the present invention;

FIG. 17 shows a medical system configured to facilitate intravascular access to the renal artery and deliver renal cryogenic denervation therapy to nerves and ganglia primarily at an ostial region of the renal artery that contribute to renal sympathetic nerve activity in accordance with embodiments of the present invention; and

FIG. 18 is a cross-section of a catheter portion of a cryoballoon catheter showing a lumen arrangement in accordance with embodiments of the present invention.

While the invention is amenable to various modifications and alternative forms, specifics thereof have been shown by way of example in the drawings and will be described in detail. It is to be understood, however, that the intention is not to limit the invention to the particular embodiments described. On the contrary, the intention is to cover all modifications, equivalents, and alternatives falling within the scope of the invention as defined by the appended claims.

### DETAILED DESCRIPTION

In the following description, references are made to the accompanying drawings which illustrate various embodiments of the invention. It is to be understood that other embodiments may be utilized, and structural and functional changes may be made to these embodiments without departing from the scope of the present invention.

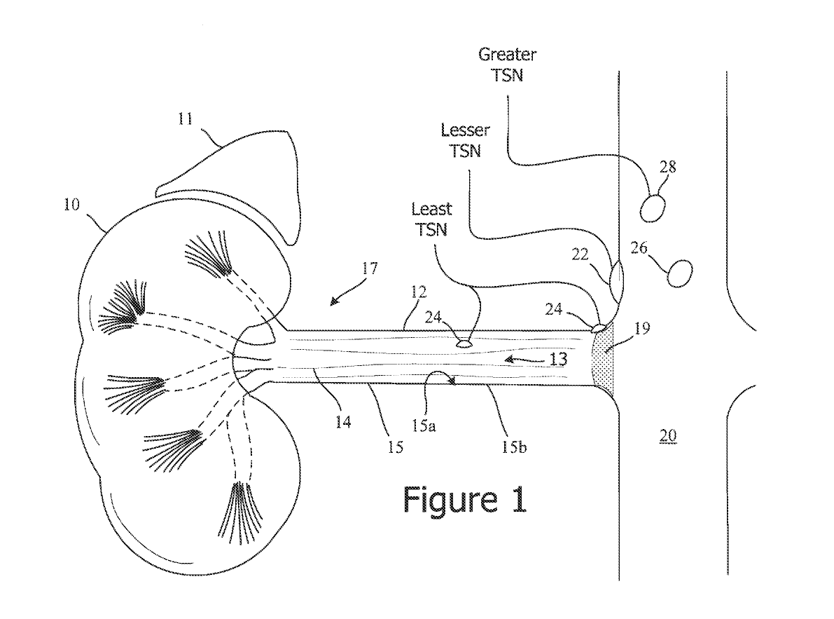

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the present invention. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

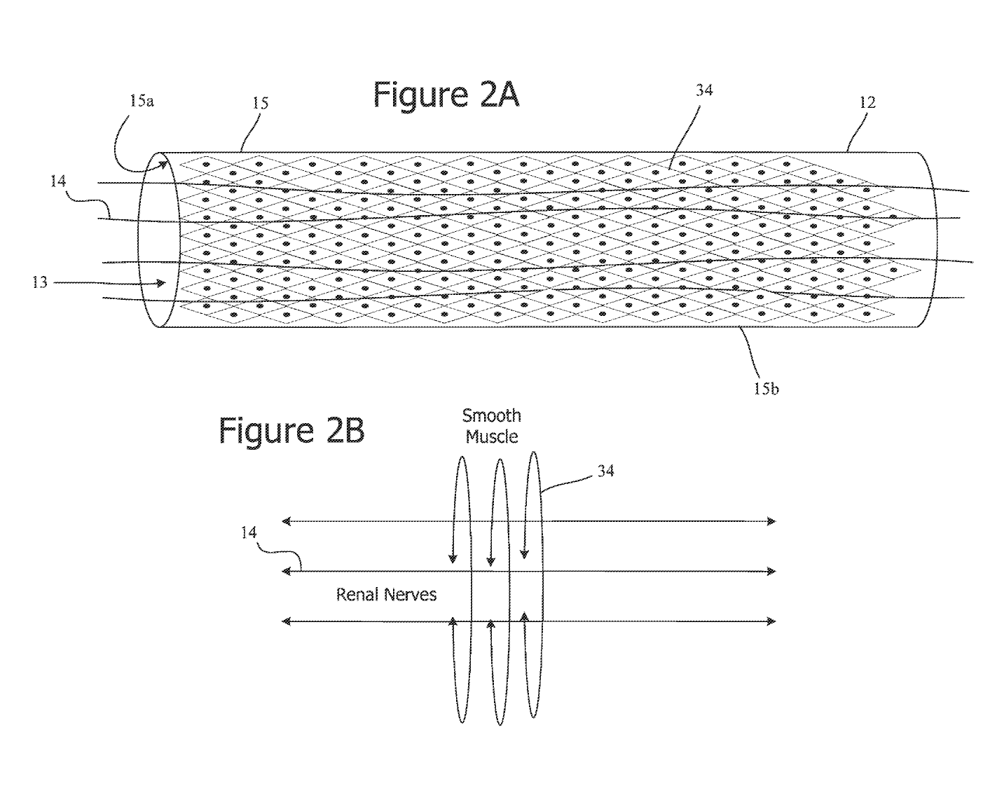

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

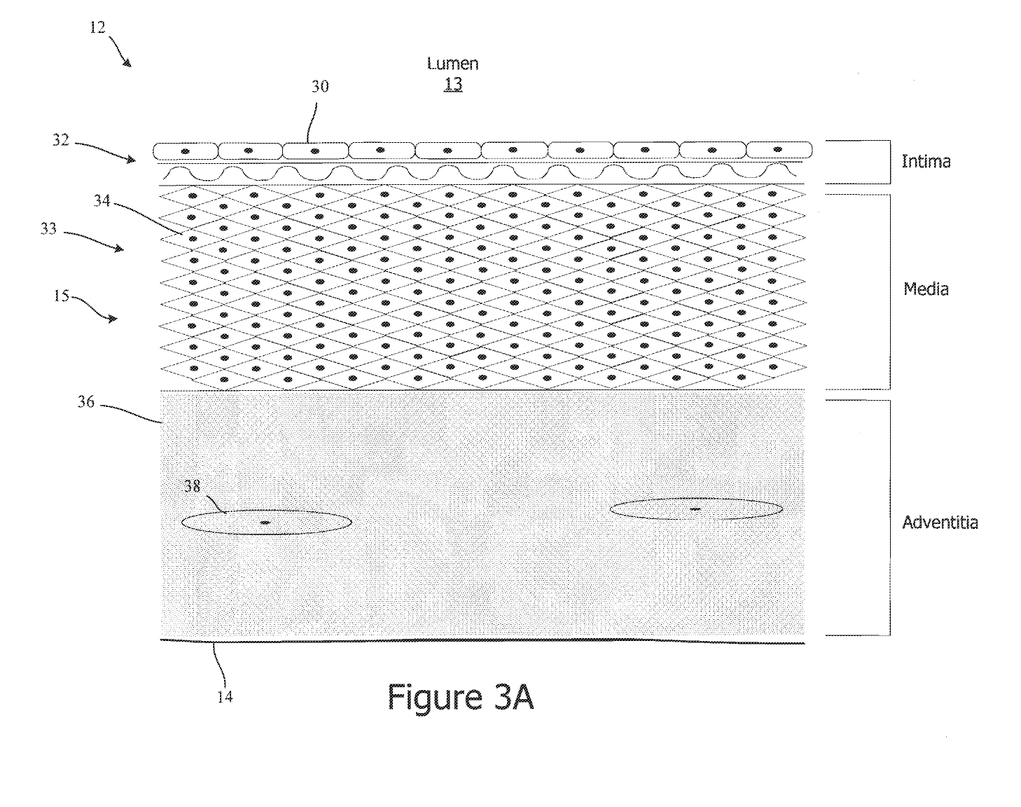

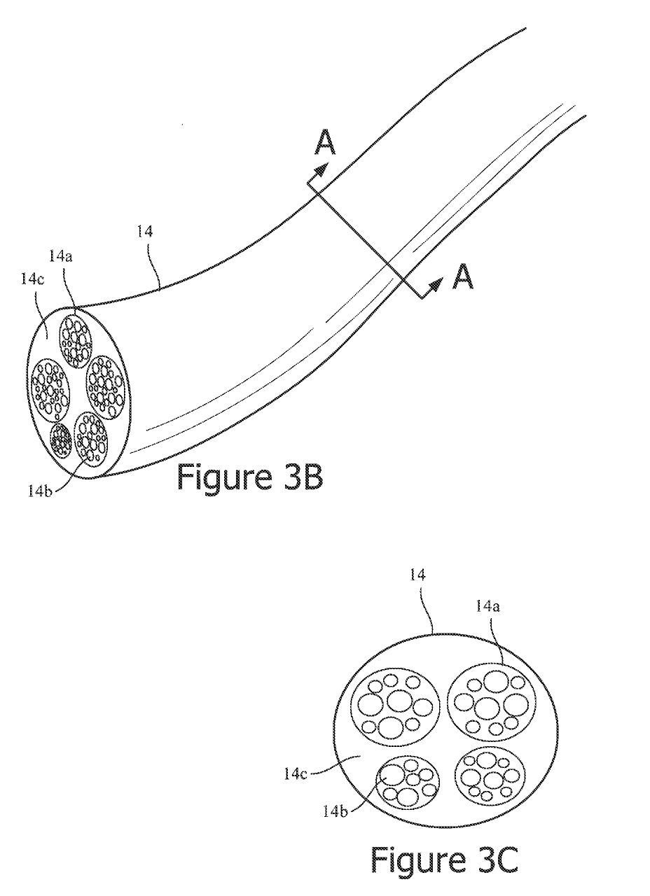

A focal location for renal innervation is the ostia 19 of the renal arteries 12. The ostium 19 of the right renal artery 12 is shown generally in FIG. 1 as the hatched region of renal vasculature at the entrance of the renal artery 12. Postganglionic nerve fibers arising from renal ganglia innervate the renal arteries 12 along a path that includes the ostia 19. FIGS. 3B and 3C illustrate various components of a renal nerve 14, a more detailed discussion of which is provided hereinbelow in the context of subjecting the nerve 14 to cryotherapy in order to reduce, and preferably irreversibly terminate, renal sympathetic nerve activity in accordance with embodiments of the present invention.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12 including the ostium 19, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner from the ostium 19 along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

FIG. 3A shows a segment of a longitudinal cross-section through a renal artery, and illustrates various tissue layers of the wall 15 of the renal artery 12, which includes the ostium 19 (best seen in FIG. 1) of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing. A renal nerve 14 is shown proximate the adventitia 36, passing into the renal artery 12 via the ostium 19, and extending longitudinally along the renal artery wall. The main trunk of the renal nerves 14 generally lies in or on the adventitia of the renal artery, with certain branches coursing into the media to enervate the renal artery smooth muscle.

Embodiments of the present invention are directed to apparatuses and methods for delivering a cryogen primarily to an ostium of a renal artery in order to modify, disrupt, or terminate renal sympathetic nerve activity. Other embodiments are directed to apparatuses and methods for delivering a cryogen primarily to an ostium of a renal artery and secondarily to a portion of the renal artery wall in order to modify, disrupt, or terminate renal sympathetic nerve activity. Preferred embodiments are those that deliver a cryogen to the ostium of a renal artery and optionally also to a renal artery wall that irreversibly terminates renal sympathetic nerve activity.

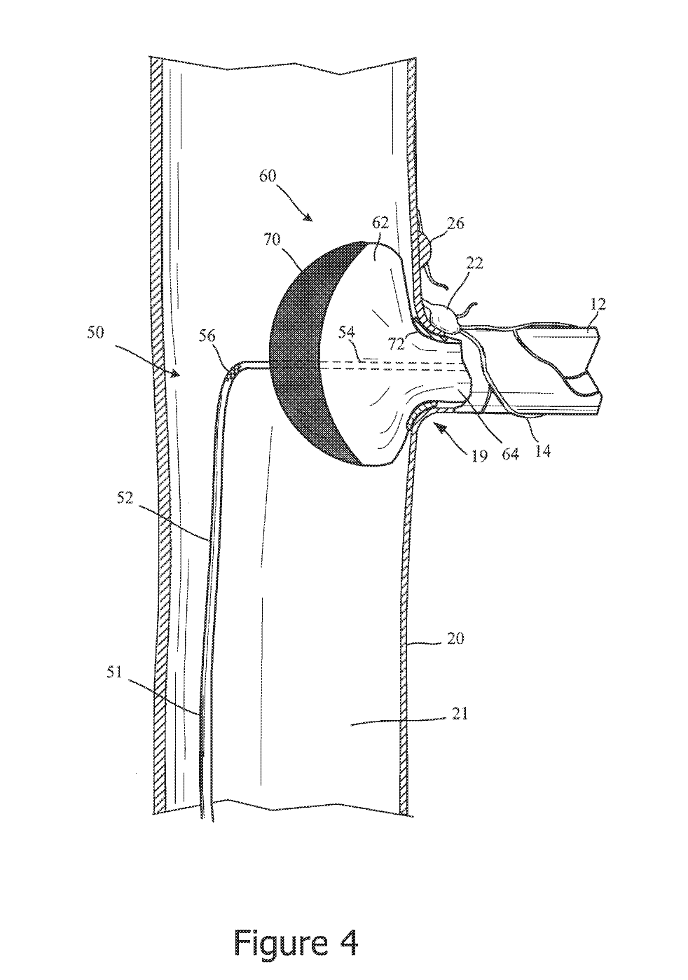

A representative embodiment of an apparatus configured to modify, disrupt, or terminate renal sympathetic nerve activity using a cryogen in accordance with the present invention is shown in FIG. 4. FIG. 4 illustrates a cryotherapy balloon catheter 50, also referred to herein as a cryoballoon catheter, in accordance with embodiments of the present invention. The cryoballoon catheter 50 includes a cryoballoon 60 provided at a distal end 54 of a catheter 51 and fluidly coupled to a cryogen source (not shown). Cryogenic fluid is delivered to the cryoballoon 60 through a supply lumen provided in the catheter 51. The cryogenic fluid, when released inside the cryoballoon 60, undergoes a phase change that cools the treatment portion of the cryoballoon 60 by absorbing the latent heat of vaporization from the tissue surrounding the cryoballoon 60, and by cooling of the vaporized gas as it enters a region of lower pressure inside the cryoballoon 60 (the Joule-Thomson effect).

As a result of the phase change and the Joule-Thompson effect, heat is extracted from the surroundings of the cryoballoon 60, thereby cooling the treatment portion of the cryoballoon 60 and aortal/renal tissue that is in contact with the treatment portion of the cryoballoon 60. The gas released inside the cryoballoon 60 may be exhausted through a separate exhaust lumen provided in the catheter 51. The pressure inside the cryoballoon 60 may be controlled by regulating one or both of a rate at which cryogenic fluid is delivered and a rate at which the exhaust gas is extracted.

It has been shown experimentally that at sufficiently low temperatures, the blood in contact with the cryoballoon's treatment portion will freeze, thereby acting as a thermally conducting medium to conduct heat away from adjacent blood, and the tissue at the ostium 19 and renal artery 12. The diameters and insulating properties of the cryoballoon 60 can be designed such that the ostium 19 is the primary target for treatment, and the middle region of the renal artery 12 may be a secondary target for treatment. Cryogenically treating the middle region of the renal artery 12 reduces the adverse impact on the distal and proximal portions of the renal artery 12. For example, the ostial and arterial balloons 62, 64 can be designed such that only partial contact with the renal artery wall is permitted and insulating material is placed elsewhere in order to reduce and control the region(s) that are subject to cryotherapy. Under-sizing the cryoballoon 60 can serve to reduce physical vessel trauma, which can be achieved by use of compliant materials in the construction of the cryoballoon 60.

FIG. 4 shows a cryoballoon catheter 50 in a deployed (inflated) configuration at the ostium 19 of a renal artery 12. The cryoballoon 60 includes an ostial balloon section 62, also referred to herein as an ostial balloon, and an arterial balloon section 64, also referred to herein as an arterial balloon. In some embodiments, an alignment element 72 is provided proximate a transition region of the cryoballoon 60, between the ostial and arterial balloons 62, 64. The alignment element 72 is preferably configured to facilitate proper positioning of the cryoballoon 60 at the renal artery during cryoballoon deployment.

The alignment element 72 may be a feature integral to the cryoballoon 60 (e.g., a thickened wall section or encapsulated elastic coupling element) or a separate element that is bonded, welded or otherwise affixed at the transition region of the cryoballoon 60. In some configurations, the alignment element 72 extends circumferentially around the transition region of the cryoballoon 60. In other configurations, an alignment element 72 is situated at one or more discrete locations (e.g., discontinuous locations) at or around the transition region of the cryoballoon 60.

For example, one or more alignment elements 72 may be situated at each of an inferior (lower) portion and a superior (upper) portion of the transition region of the cryoballoon 60, so as to contact inferior and superior portions of the ostium 19 of the renal artery 12, respectively. FIG. 4 illustrates such a configuration, in which the ostial balloon 62 abuts the ostium 19 with an alignment element 72 disposed immediately adjacent to, and in direct contact with, the ostial tissue. In other configurations, one or more alignment elements 72 may be situated at an inferior portion (but not at a superior portion) of the of the transition region of the cryoballoon 60, so as to contact the inferior portion of the ostium 19. In this configuration, the superior portion of the outer wall of the ostial balloon abuts directly against the ostial tissue.

The alignment element 72 is preferably formed of a thermally conductive material and/or has the property of moderating thermal conduction at the ostial treatment site. In some embodiments, the alignment element 72 is configured as a primary cryotherapy delivery component for cryogenically treating the ostium 72 of the renal artery 12. The alignment element 72 may be implemented to provide a thermal conduction path between a cryogen contained within the ostial balloon 62 (or catheter 51) and ostial tissue at the renal artery 12. In other configurations, the alignment element 72 may be implemented to include one or more hollow sections that receive a cryogen contained within the ostial balloon 62 (or catheter 51), providing direct cryotherapy to ostial tissue at the renal artery 12.

As is depicted in FIG. 4, the arterial balloon 64 is shown extending into the renal artery 12 and is preferably in contact with the inner wall of the renal artery 12. The ostial balloon 62 is shown abutting the ostium 19 of the renal artery 12 and surrounding tissue of the abdominal aorta 20. Preferably, when in abutment with the ostium 19, the ostial balloon 62 is configured to deliver cryotherapy to a region of vasculature that encompasses renal nerves and ganglia at or near the ostium 19, including the aorticorenal ganglion 22. In some configurations, the ostial balloon 62 may be configured to deliver cryotherapy to a region of aortal/renal vasculature that encompasses renal nerves at or near the ostium 19, the aorticorenal ganglion 22, and the superior mesenteric ganglion 26.

The cryoballoon 62 shown in FIG. 4 is primarily constructed to deliver cryotherapy to the ostial region 19 of the aortal/renal vasculature. In some embodiments, the arterial balloon 64 is constructed primarily for facilitating proper positioning of the ostial balloon 62 in abutting contact with the ostium 19 of the renal artery 12. In this case, the arterial balloon 64 is configured primarily as a stabilizing or anchoring balloon, and may be constructed as a non-compliant balloon, similar to a dilation balloon. Alternatively, the arterial balloon 64 may be constructed as a compliant balloon and configured to stabilize or anchor the ostial balloon 62 in proper position. In such configurations, only the ostial balloon 62 (and/or the alignment element 72) is provided with cryotherapy delivery elements.

In accordance with other embodiments, both the ostial balloon 62 and the arterial balloon 64 include cryotherapy delivery elements. In some embodiments, the ostial balloon 62 and the arterial balloon 64 are constructed as compliant balloons. In other embodiments, the ostial balloon 62 is constructed as a compliant balloon and the arterial balloon 64 is constructed as a non-compliant balloon. As will be discussed hereinbelow, the ostial balloon 62 may be constructed as a single balloon or have a multiple balloon construction. In a multiple balloon implementation, an inner ostial balloon contains a cryogen and an outer ostial balloon is inflatable using a passive fluid, such as saline.

At least the ostial balloon 62 (and both ostial and arterial balloons 62 and 64 in some embodiments) is constructed as a very low pressure system and/or can be undersized in comparison to dimensions of the renal artery 12. The cryoballoon 60 is preferably constructed as a compliant balloon as is known in the art. For example, cryoballoon 60 may comprise a compliant material configured to enable the cryoballoon 60 to inflate under a very low pressure, such as about 1 to 2 pounds per square inch (PSI) or less (e.g., 0.5 PSI or less) above an ambient pressure that is adjacent to and outside the cryoballoon 60. The compliancy of cryoballoon 60 readily allows at least the ostial balloon 62 to conform to irregularities in the shape of the ostium 19 and surrounding tissue of the aortal/renal vasculature, which results in more efficient delivery of cryotherapy to the target tissue (i.e., renal nerve fibers and renal ganglia).

All or a portion of the cryoballoon 60 (e.g., at least the ostial balloon 62, or both ostial and arterial balloons 62 and 54 in some embodiments) may be made of a highly compliant material that elastically expands upon pressurization. Because the cryoballoon 60 elastically expands from a deflated state to an inflated state, the cryoballoon 60 has an extremely low profile in the deflated state when compared to non-compliant or semi-compliant balloons. Use of high compliance materials in the construction of the cryoballoon 60, in combination with a hinge mechanism 56 built into the catheter 51, provides for enhanced efficacy and safety when attempting to navigate a cryoballoon catheter 50 of the present invention through a nearly 90 degree turn from the abdominal aorta 20 into the ostium 19 of the renal artery 12.

Suitable materials for constructing all or a portion of the cryoballoon 60 include thermoplastic or thermoplastic elastomers, rubber type materials such as polyurethanes, natural rubber, or synthetic rubbers. The resulting balloon may be crosslinked or non-crosslinked. Other suitable materials for constructing all or a portion of the cryoballoon 60 include silicone, urethane polymer, low durometer PEBAX, or an extruded thermoplastic polyisoprene rubber such as a low durometer hydrogenated polyisoprene rubber. These and other suitable materials may be used individually or in combination to construct the cryoballoon 60. Details of various materials suitable for constructing a cryoballoon 60 are disclosed in commonly owned U.S. Patent Publication No. 2005/0197668, which is incorporated herein by reference.

With continued reference to FIG. 4, a proximal portion of the ostial balloon 62 may include an insulated section 70 to prevent freezing of blood in the main lumen 21 of the abdominal aorta 20 that comes into contact with the ostial balloon 62. Provision of an insulated proximal section 70 advantageously reduces the likelihood of injury to non-targeted treatment sites, such as the opposite side of the main lumen 21 of the abdominal aorta 20. The insulated proximal section 70 may be an insulating coating or combination of insulating coatings that are deposited by manually painting the coating, dipcoating, spraying, solvent casting, or using other known application techniques. In a cryoballoon configuration than employs dual ostial balloon, for example, an insulated proximal section 70 may be provided as an insulating gas layer developed between balloon materials. In other configurations, an insulated proximal section 70 may be fabricated by applying (e.g., adhering) an additional polymer layer to the ostial balloon 62 after the ostial balloon 62 is molded. These and other techniques may be used individually or in combination to construct an ostial cryoballoon 62 having an insulated proximal section 70.

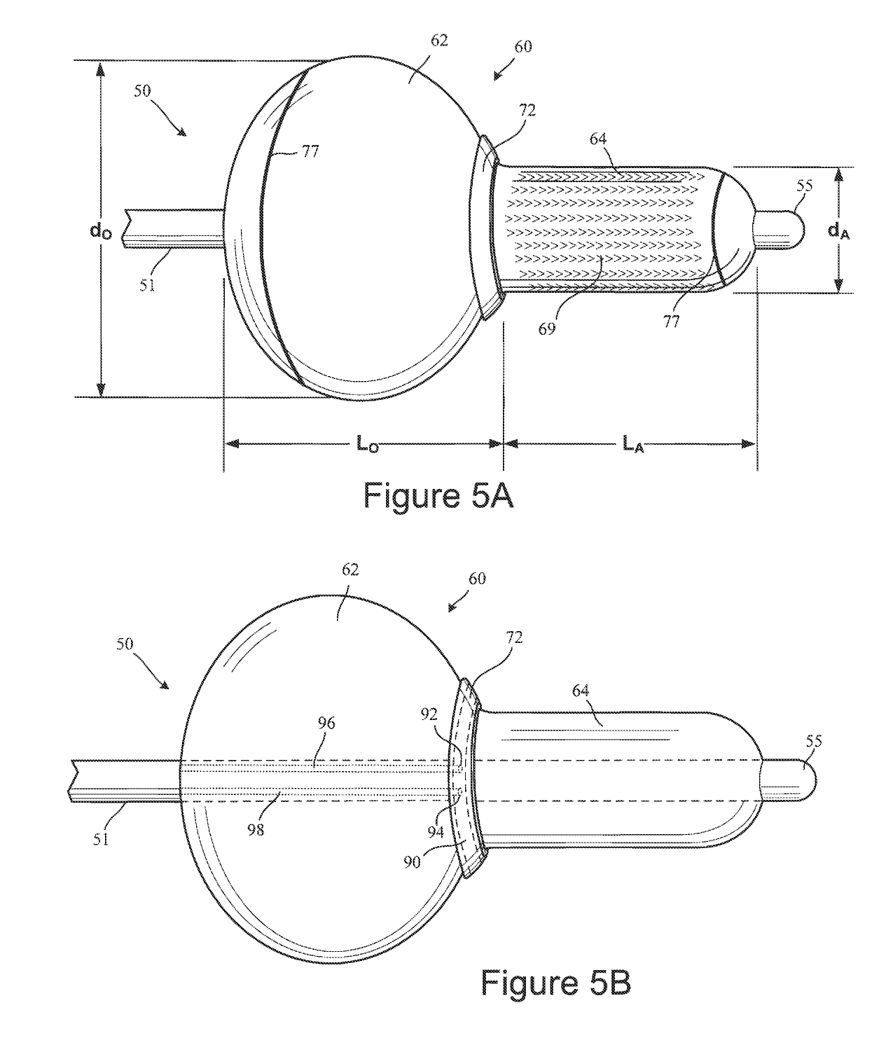

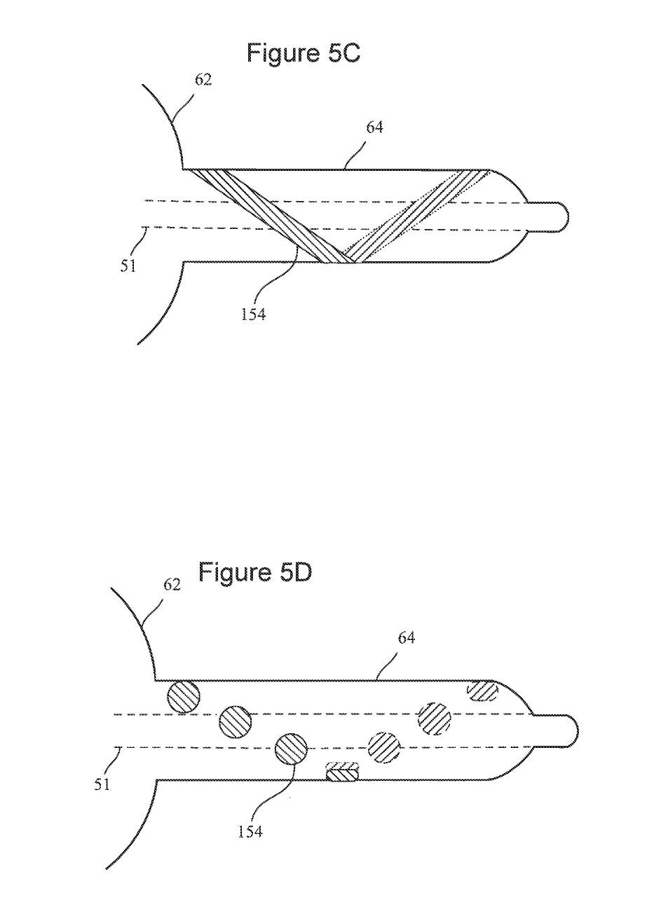

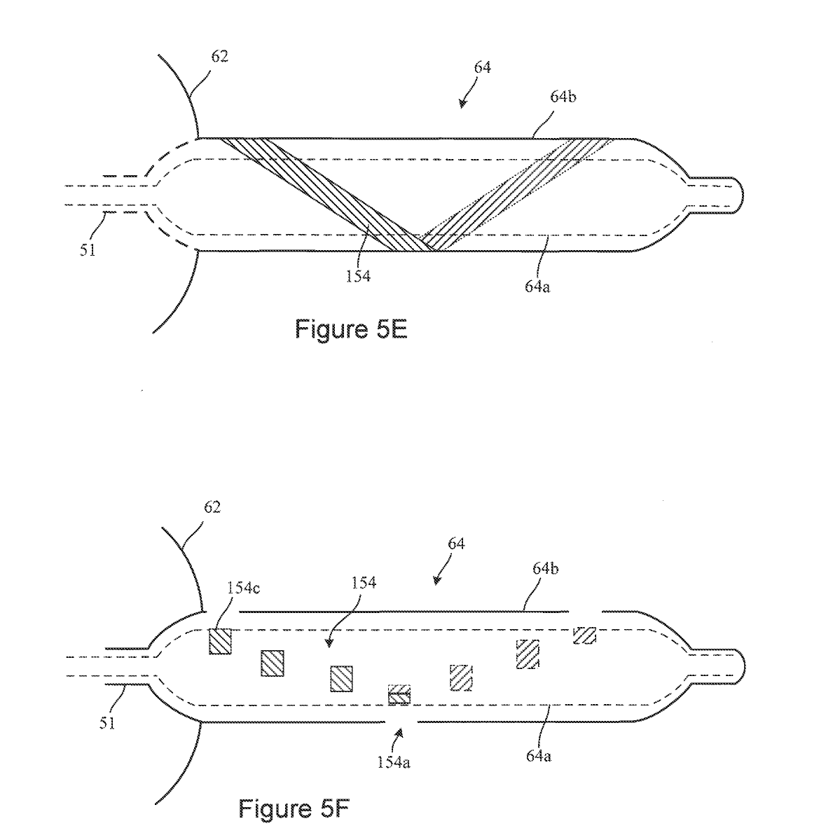

FIG. 5A illustrates the distal portion of a cryoballoon catheter 50 configured for deployment at the ostium, and within the lumen, of the renal artery in accordance with embodiments of the present invention. The cryoballoon catheter 50 shown in FIG. 5A includes a cryoballoon 60 comprising a distal arterial balloon 64, a proximal ostial balloon 62 and an alignment element 72 provided at a transition location between the arterial and ostial balloons 64 and 62. The cryoballoon 60 is disposed at the distal portion 54 of the catheter, which is shown to have a closed lumen at the catheter's tip 55. It is noted that, in an alternative embodiment, the catheter's tip 55 may incorporate an open lumen to facilitate longitudinal displacement of a guide wire for over-the-wire delivery of the cryoballoon 60 into the renal artery 12. In the closed lumen embodiment shown in FIG. 5A, the added complexity and deployment time associated with over-the-wire delivery is avoided by incorporation of a hinge mechanism (shown in other figures) in the distal portion 54 of the catheter.

In FIG. 5A, the cryoballoon 60 is illustrated in an inflated configuration. The cryoballoon 60 can be implemented to achieve desired expansion profiles for each of the ostial balloon 62 and the arterial balloon 64. The materials, wall thicknesses, diameters, and other dimensions and construction features can be judiciously selected to achieve desired longitudinal and radial expansion characteristics of the ostial and arterial balloons 62, 64. For example, the ostial balloon 62 can be constructed to provide preferential expansion of its diameter, do, relative to expansion of its longitudinal dimension, Lo. For example, the ratio of do/Lo expansion can range between about 2:1 and about 6:1. This preferential radial expansion profile of the ostial balloon 62 serves to reduce the volume of the proximal portion of the ostial balloon 62 within the aorta, thereby reducing occlusion of blood flow within the aorta.

By way of further example, the arterial balloon 64 can be constructed to provide preferential expansion of its longitudinal dimension, L_{A}, dimension relative to expansion of its diameter, d_{A}. For example, the arterial balloon 64 may be configured to expand along its longitudinal dimension, L_{A}, by up to about 400% of its original length, while the diameter, d_{A}, remains about the same size or expands up to about 20% of its original size. This preferential longitudinal expansion profile of the arterial balloon 64 allows for a more compact delivery device which would aid in deliverability. This preferential longitudinal expansion profile of the arterial balloon 64 also serves to reduce the circumferential pressure exerted on the renal artery wall by increasing the surface area of contact between the arterial balloon 64 and the renal artery wall.

In some embodiments, the diameter, do, of the cryoballoon 60 at the balloon's proximal end is between about 10% to about 100% greater than the diameter, d_{A}, of the cryoballoon 60 at the balloon's distal end. In other embodiments, the diameter, do, of the cryoballoon 60 at the balloon's proximal end is between about 10% to about 400% greater than the diameter, d_{A}, of the cryoballoon 60 at the balloon's distal end. In further embodiments, the diameter, d_{O}, of the cryoballoon 60 at the balloon's proximal end is at least 200% greater than the diameter, d_{A}, of the cryoballoon 60 at the balloon's distal end. These representative diameter relationships may be applicable to the cryoballoon 60 in a deflated configuration or when inflated at a therapeutic pressure.

According to some embodiments, a traction feature may be included on the arterial balloon 64. Various fraction feature implementations are contemplated for improving the traction between the arterial balloon 64 and renal artery wall tissue. A traction feature is preferably situated on, applied to, or incorporated in the outer surface of the arterial balloon 64 to reduce slippage or undesirable movement of the arterial balloon 64 within the renal artery 12, which could otherwise result in dislodgment of the arterial balloon 64 from a desired location within the renal artery 12. For example, and assuming the cryoballoon 60 is deployed as shown in FIG. 4, increasing the inflation pressure within the ostial balloon 62 can generate a force against the ostium region 19 of the renal artery 12 that tends to displace the arterial balloon 64 in a proximal direction.

Employment of a traction feature, such as a gripping feature shown in FIG. 5A, can provide a surface that aids in maintaining the position of the arterial balloon 64 within the renal artery 12 at a desired location and reducing slippage of the arterial balloon 64 from the desired location. Inclusion of one or more traction features provides for enhanced control when positioning the arterial balloon 64 and more precise control of cryoablation therapy delivery to target innervated renal tissue (e.g., perivascular renal tissue).

The representative embodiment illustrated in FIG. 5A shows one type of traction feature that can be implemented to enhance gripping of renal artery wall tissue by the arterial balloon 64 when deployed within the renal artery 12. The outer surface of the arterial balloon 64 shown in FIG. 5A includes a gripping region 69 defined by a textured surface, which may be a series of ridges, bumps, or projections having a desired shape and size. The textured gripping surface 69 can be formed or defined in any suitable manner. For example, the textured gripping surface 69 can be formed by scoring, grinding, scuffing, or otherwise altering the outer surface of the arterial balloon 64 or other member situated over the arterial balloon's outer surface. The pattern of textured surface 69 may vary and can be random, regular, intermittent, or any other suitable pattern. Details of various traction features that can be incorporated in or on an arterial balloon 64 according to various embodiments are described in commonly owned U.S. Pat. No. 7,566,319, which is incorporated herein by reference.

The cryoballoon catheter 50 can be designed such that pre-inflation of the cryoballoon 60 with a syringe using saline or similar media can partially inflate the proximal ostial balloon 62 in order to seat the ostial balloon 62 against the ostium 19 of the renal artery 12 prior to applying the cryotherapy. Alternatively, a small volume of cryogenic fluid may be injected into the cryoballoon 60 for pre-inflation purposes (e.g., at a rate to slightly inflate the cryoballoon 60 but insufficient to implicate Joule-Thompson effect cooling). After positioning the ostial balloon 62 against the ostium 19 of the renal artery 12, cryogenic fluid is injected into the cryoballoon 60 to controllably initiate cryotherapy, causing both the ostial balloon 62 and the distal arterial balloon 64 to inflate. This can be accomplished, for example, by constraining the region near the transition location 72 between the ostial balloon 62 and the arterial balloon 64, such as by using balloon crimping methods, manual restrictions, folding methods, and/or physical flow restrictions. In some embodiments, the cryoballoon catheter 50 may comprise multiple balloons, some of which are configured for pressurization using a cryogenic fluid, while others are configured for pressurization using saline or other passive fluid. A pre-inflation technique discussed above may be used in single- and multiple-balloon cryotherapy balloon catheters of the present invention.

Marker bands 77 can be placed on one or multiple parts of the ostial and arterial balloons 62, 64 to enable visualization during the procedure. Other portions of the cryoballoon 60, such as the alignment element 72, may include a marker band, as can one or more portions of the catheter shaft 51 (e.g., at the hinge mechanism 56). The marker bands 77 may be solid or split bands of platinum or other radiopaque metal, for example. Radiopaque materials are understood to be materials capable of producing a relatively bright image on a fluoroscopy screen or another imaging technique during a medical procedure. This relatively bright image aids the user of the cryoballoon catheter 50 in determining its location.

As was discussed previously, the alignment element 72 is preferably formed of a thermally conductive material and/or has the property of moderating thermal conduction at the ostial treatment site. In the embodiment shown in FIG. 5B, the alignment element 72 is configured as a primary cryotherapy delivery element for cryogenically treating the ostium 72 of the renal artery 12. The alignment element 72 of FIG. 5B is preferably hollow and includes an inlet port 92 and an outlet port 94. A circulation path is defined within the hollow portion of the alignment element 72 between the inlet and outlet ports 92, 94.

The inlet port 92 is fluidly coupled to a supply lumen 96 of the catheter 51, and the output port 94 is fluidly coupled to an exhaust lumen 98 of the catheter 51. A cryogenic fluid is delivered to the alignment element 72 from a cryogen source via the supply lumen 92 and inlet port 92, and exhaust gas (or liquid) is removed from the alignment element 72 via the outlet port 94 and exhaust lumen 98. In this configuration, the alignment element 72 provides direct cryotherapy to ostial tissue at the renal artery 12. In some configurations, the alignment element 72 may be built into the distal portion of the ostial balloon 62 or maybe a separate component that is affixed to the balloon arrangement subsequent to fabrication of the ostial and arterial balloons 62, 64.

The arterial balloon 64 of the cryoballoon arrangement 60 may be constructed to include cryotherapy elements that are arranged in accordance with a predetermined pattern for purposes of delivering patterned cryotherapy to the inner wall of the renal artery 12. FIGS. 5C and 5D illustrate two embodiments of a patterned cryotherapy arterial balloon 64. The cryoballoons 60 shown in FIGS. 5C and 5D each comprise a balloon arrangement that incorporates a predefined treatment pattern 154. The treatment pattern 154 of the arterial balloon 64 may be fashioned as a separate component from the arterial balloon 64 and subsequently affixed thereto (e.g., a patterned sleeve or sheath) or formed as in integral element of the arterial balloon 64. The patterned arrangement 154 of the arterial balloon 64 may comprise one or more surface structures or treatment features, surface discontinuities, voids or apertures, or combinations of these and other features. A cryogenic fluid is communicated to the treatment pattern 154 of the arterial balloon 64 to deliver cryogenic denervation therapy to the renal nerves innervating the renal artery 12.

According to some embodiments, the outer surface of the arterial balloon 64 incorporates material with a relatively low thermal conductivity (e.g., thermally insulating material) that forms the main body of the arterial balloon 64. The treatment pattern 154 or pattern segments 154 are formed from relatively high thermally conductive material. In other embodiments, an inner layer of the arterial balloon 64 may incorporate a polymeric composite material with a low thermal conductivity, and the outer portion of the arterial balloon 64 may incorporate a patterned or apertured layer comprising a polymeric composite material with a low thermal conductivity. In such embodiments, regions of the inner layer with high thermal conductivity are exposed for thermally treating renal ostial and arterial tissue through apertures of the outer layer with low thermal conductivity.

FIGS. 5E and 5F illustrate embodiments of arterial balloons 64 that include dual balloon arrangements 64a, 64b. In some embodiments, as shown in FIG. 5E, an outer balloon 64b of the arterial balloon 64 incorporates a treatment pattern 154 configured to facilitate delivery of a cryogenic denervation therapy to the renal artery 12. An inner balloon 64a serves as a biasing balloon that, when inflated, expands and forces at least the treatment pattern arrangement 154 of the outer balloon 64b against the inner wall of the renal artery 12. The inner balloon 64a may be controllably pressurized using saline or other passive fluid. A cryogen is communicated to the treatment pattern arrangement 154 via a conduit of the outer balloon 64b or the inner balloon 64a. The cryogen may also be used to pressurize the outer balloon 64b or another fluid may be used, such as saline.

In some embodiments, the outer balloon 64b may have a generally cylindrical outer profile. In other embodiments, the profile of the outer balloon 64b may have a fluted, wave, or other complex shape that is configured to contact a vessel's inner wall at longitudinally and circumferentially spaced-apart locations. Each of these contact locations of the outer balloon 64b preferably incorporates a treatment pattern segment or segments, and the effective coverage area (e.g., area of pattern structure or void) of the treatment pattern segments preferably completes at least one revolution or turn of the outer balloon 64b.

According to other embodiments, as shown in FIG. 5F, the outer balloon 64b of the arterial balloon 64 incorporates a treatment pattern 154 comprising voids or apertures 154a. An inner balloon 64a incorporates a thermally active treatment pattern 154c that is shown to be in alignment with the voids or apertures 154a of the outer balloon 64b. Alternatively, the inner cryoballoon 64a need not be patterned. The inner balloon 64a also serves as a biasing balloon that, when inflated, expands and forces at least the treatment pattern 159c of the inner balloon 64a against or in proximity with the inner wall of the renal artery 12. The inner balloon 64a may be controllably pressurized using saline or by the cryogen that is fluidly or thermally coupled to the thermally active treatment pattern 154c. The outer balloon 64b may be controllably pressurized using saline or other passive fluid. Additional details of patterned cryogenic balloons and associated components that may be incorporated into a cryotherapy balloon catheter of the present invention are disclosed in commonly owned U.S. Patent Publication No. ______, and receiving U.S. Provisional Ser. No. 61/291,480 filed on Dec. 31, 2009 under Attorney Docket No. BCV.006.P1 and entitled “Patterned Denervation Therapy For Innervated Renal Vasculature,” which is incorporated herein by reference.

A cryoballoon that incorporates a predetermined pattern of thermally active material or regions encompassing at least one complete turn or revolution of the cryoballoon advantageously facilitates a “one-shot” denervation therapy of the ostium 19 and renal artery 12 in accordance with embodiments of the present invention. The term “one-shot” treatment refers to treating the entirety of a desired portion of innervated vascular tissue (e.g., ostium 19 of the renal artery, renal artery 12) without having to move the cryoballoon arrangement to other vessel locations in order to complete the treatment procedure (as is the case for a step-and-repeat denervation therapy approach).

A one-shot treatment approach of the present invention advantageously facilitates delivery of denervation therapy that treats at least one location of each nerve fiber passing through the ostium 19 of the renal artery 12 and, in some embodiments, also those extending along the renal artery 12, without having to reposition the cryoballoon catheter 50 during denervation therapy delivery. Embodiments of the present invention allow a physician to position a cryoballoon catheter 50 at a desired vessel location, and completely treat innervated renal vasculature without having to move the cryoballoon catheter 50 to a new vessel location. A one-shot treatment approach of the present invention also facilitates delivery of cryogenic denervation therapy that treats one or more ganglia proximate the ostium 19 of the renal artery 12 without having to reposition the cryoballoon catheter 50 during denervation therapy delivery. It is to be understood that devices and methods that utilize a cryoballoon catheter 50 of the present invention provide advantages and benefits other than facilitating one-shot treatment of a vessel or ganglion, and that cryoballoon patterning that enables one-shot vessel or ganglion treatment is not a required feature in all embodiments.

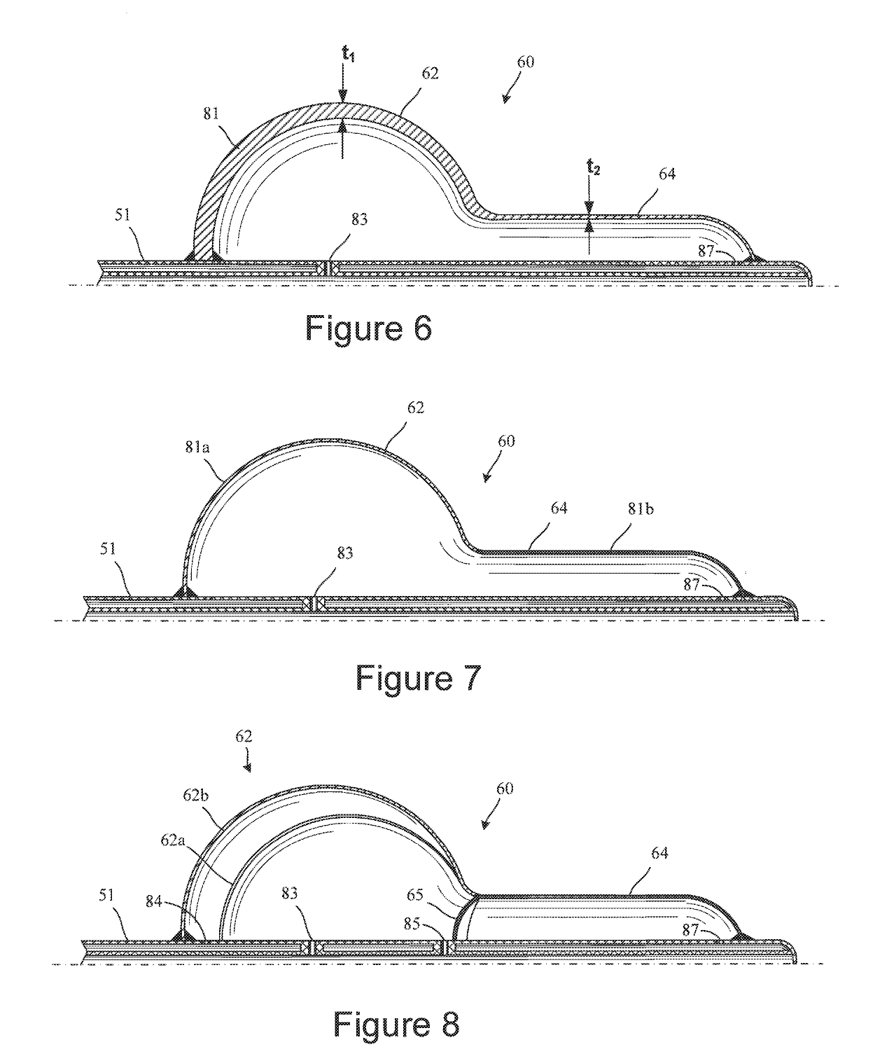

FIG. 6 is a cross-section of a cryoballoon 60 in accordance with embodiments of the present invention. The cryoballoon 60 shown in FIG. 6 is constructed to have a balloon wall 81 that varies in thickness along its longitudinal axis. This variation in balloon wall thickness provides for varying balloon diameters relative to the longitudinal axis of the cryoballoon 60, which are more pronounced when the cryoballoon 60 is inflated. In this illustrative example, the balloon wall 81 at a proximal section 62 of the cryoballoon 60 has a thickness, t1, that is greater than a thickness, t2, of the balloon wall 81 at the cryoballoon's distal section 64. The thickness of the balloon wall 81 is shown in FIG. 6 to vary continuously relative to the longitudinal axis of the cryoballoon 60. Changes in balloon wall thickness can be continuous (as shown in FIG. 6) or occur in a step-wise or other fashion to achieve desired balloon expansion characteristics. The balloon wall thickness can vary for each of the ostial balloon 62 and the arterial balloon 64, and need not have a continuously thinning or thickening profile as depicted in FIG. 6. Further, the lengths of the proximal and distal balloons 62, 64 can be the same or different.

As shown in FIG. 6, the proximal portion of the cryoballoon 60 (e.g., ostial balloon 62) has a wall thickness, t₁, that is greater than a wall thickness, t₂, of the distal portion of the cryoballoon 60 (e.g., arterial balloon 64). The increased thickness in the proximal section 62 requires a greater pressure to achieve inflation relative to the distal section 64. Depending on the construction of the cryoballoon 60, it may be desirable to have the distal section 64 inflate more easily than the proximal section 62. The expansion profile of the cryoballoon 60 allows the arterial balloon 64 to expand into the renal artery 12 prior to full inflation of the ostial balloon 62, which provides for enhanced positioning and stabilization of the ostial balloon 62 at the ostium 19 of the renal artery 12.

In this implementation, the amount of pressure necessary to achieve at least partial inflation of the distal section 64 is insufficient to fully inflate the proximal section 62, allowing for preferential expansion of the arterial balloon 64 into the renal artery 12 relative to expansion of the ostial balloon 62 within the aorta 20. Once the distal portion 64 of the cryoballoon 60 is inflated to the desired pressure or diameter, injection of additional pressurized fluid causes the pressure in the cryoballoon 60 to increase, resulting in further inflation and expansion of the proximal section 62 within the aorta 20. The dimensions of the arterial balloon 64 preferably allow for longitudinal expansion within the renal artery 12 during continued pressurization and expansion of the ostial balloon 62, with adequate space allotted for over-pressurization situations.

In other implementations, it may be desirable to provide equal or greater radial expansion of the ostial balloon 62 during balloon pressurization relative to radial and/or longitudinal expansion of the arterial balloon. This implementation may be useful in embodiments that only employ cryotherapy elements within the ostial balloon 62, with the arterial balloon 64 used primarily as positioning/stabilization element.

It is understood that differences in thickness between the distal section 64 and proximal section 62 of the cryoballoon 60 are selected to achieve desired inflation characteristics. For example, in one embodiment, the distal section 64 is about three-quarters to one-half the thickness of the proximal section 62. In another embodiment, the distal section 64 is about one-half to one-third the thickness of the proximal section 62. In other embodiments, the distal section 64 has about the same thickness of the proximal section 62. In further embodiments, at least a section of the proximal section 62 has a thickness equal to or less than at least a section of the distal section 64. Other thickness relationships between proximal and distal balloon portions 62, 64 are contemplated.

FIG. 6 further illustrates a manifold 83 which is fluidly coupled to one or more lumens of the catheter 51. The manifold 83 may incorporate one or more supply ports and one or more exhaust ports for supplying cryogenic fluid to the cryoballoon 60 and removing exhaust gas therefrom. The manifold 83 may also incorporate one or more supply ports and one or more exhaust ports for supplying saline or other pressurizing fluid to the cryoballoon 60 (e.g., a separate inflation balloon of the cryoballoon 60) and removing the pressurizing fluid therefrom. The cryoballoon 60 may include multiple manifolds, 83 and 87, for managing distribution of cryogenic fluid and passive pressurizing fluid. Multiple manifolds 83 and 87 may also be used in configurations that employ separate ostial and arterial balloons 62, 64.

FIG. 7 is a cross-section of a cryoballoon 60 in accordance with other embodiments of the present invention. The cryoballoon 60 shown in FIG. 7 is constructed using different materials that offer different expansion characteristics. The ostial balloon 62 comprises a material 81a that differs from a material 81b of the arterial balloon 64. The material 81a of the ostial balloon 62, for example, may be more elastic or, alternatively, less elastic than the material 81b of the arterial balloon 64.

The materials used to construct the cryoballoon 60 can be selected to achieve desired expansion profiles for each of the ostial balloon 62 and the arterial balloon 64. For example, appropriate materials and thicknesses of such materials may be selected to achieve desired longitudinal and radial expansion characteristics of the ostial and arterial balloons 62, 64. It is noted that the thickness of the materials used for constructing the cryoballoon 60 may be different or the same for each material, or may vary as discussed above with reference to FIG. 6. Although the cryoballoon 60 shown in FIG. 7 is formed using two different materials 81a and 81b, it is understood that more than two materials and/or more than two sections of different materials may be used in the construction of the cryoballoon 60.

FIG. 8 is a cross-section of a cryoballoon 60 in accordance with further embodiments of the present invention. The cryoballoon 60 shown in FIG. 8 combines aspects of the cryoballoon embodiments discussed with reference to FIGS. 6 and 7. The cryoballoon 60 shown in FIG. 8 incorporates a dual ostial balloon configuration, where the ostial balloon 62 includes an inner balloon 62a and an outer balloon 62b. Each of the inner and outer balloons 62a, 62b is fluidly coupled to a separate lumen(s) of the distal end 54 of the catheter via separate manifolds 83, 84, 85. The arterial balloon 64 is fluidly coupled to a separate lumen of the catheter 51 via manifold 87.

The inner balloon 62a shown in FIG. 8 is preferably constructed to receive a cryogenic fluid from a lumen of the catheter 51 via supply and exhaust manifold 83 and 85. The outer balloon 62b is preferably constructed to receive a passive fluid, such as saline, from a separate lumen of the distal end 54 of the catheter via a manifold 84. The arterial balloon 64 is preferably constructed to receive saline or similar fluid from a separate lumen of the catheter 51 via a manifold 87. Alternatively, the arterial balloon 64 may be constructed to receive a cryogenic fluid via the manifold 87, which would include a supply port and an exhaust port, or include an additional manifold. The proximal wall 65 of the arterial balloon 64 may be excluded in an embodiment in which a common cryoballoon structure comprising inner ostial balloon 62a and arterial balloon 64 is desired.

In FIG. 8, the arterial balloon 64 comprises a material different than that of the ostial balloon 62. The inner ostial balloon 62a may comprise a material the same as, or different than, that of the outer ostial balloon 62b. The inner ostial balloon 62a may include an insulating layer to limit thermal cooling of the outer ostial balloon 62b. Alternative or additional thermal insulation between the inner and outer ostial balloons 62a and 62b may be facilitated by gas provided between the two balloons 62a, 62b.

It will be appreciated that the embodiments shown in FIGS. 6-9 are for non-limiting illustrative purposes, and that other implementations are contemplated. The materials, number of balloons, types of cryogens, and other construction particulars used to fabricate the cryoballoon catheter 50 can be selected to achieve desired mechanical and thermal characteristics.

A cryoballoon 60 of the present invention can be manufactured using various techniques, including molding techniques or solution casting methods, for example. According to one molding technique, gradient extruded tubes with a short transition length for two different proximal and distal material properties can be used. Cryoballoons 60 may be formed by combining materials with large differences in modulus or different levels of cross-linking Desired mechanical and thermal characteristics may be obtained by using materials with different properties (e.g., using filled or non-filled materials), or by use of tubes having different wall thicknesses.

Another molding technique involves forming balloons or portions of a balloon having different extruded tube wall thicknesses. A further approach involves forming different wall thickness tubes achieved after extrusion by removing a certain amount material from its outer diameter via a mechanical method, such as a grinding or laser abrasion process. Two or more different tubes having different wall thickness, material, and/or different inner/outer diameters, may be joined by forming a lap joint therebetween, such as by use of a melt process via thermal energy, laser energy, or ultrasonic energy. The resulting balloon tube can have different materials, and/or different wall thickness, and/or different inner/outer diameters to meet specified balloon shape requirements. Various balloon parts can be extruded or injection molded.

According to a representative solution casting technique, the balloons of a cryoballoon 60 can be manufactured with solution casting using thermoplastic or a thermoplastic elastomer, or rubbery type materials, such as polyurethanes, natural rubber, synthetic rubbers, silicone, or other appropriate material (e.g., low durometer material at least for the ostial balloon). The resulting balloon may be crosslinked or non-crosslinked. Other thin-wall fabrication techniques may be used to construct a cryoballoon 60 in accordance with embodiments of the present invention.

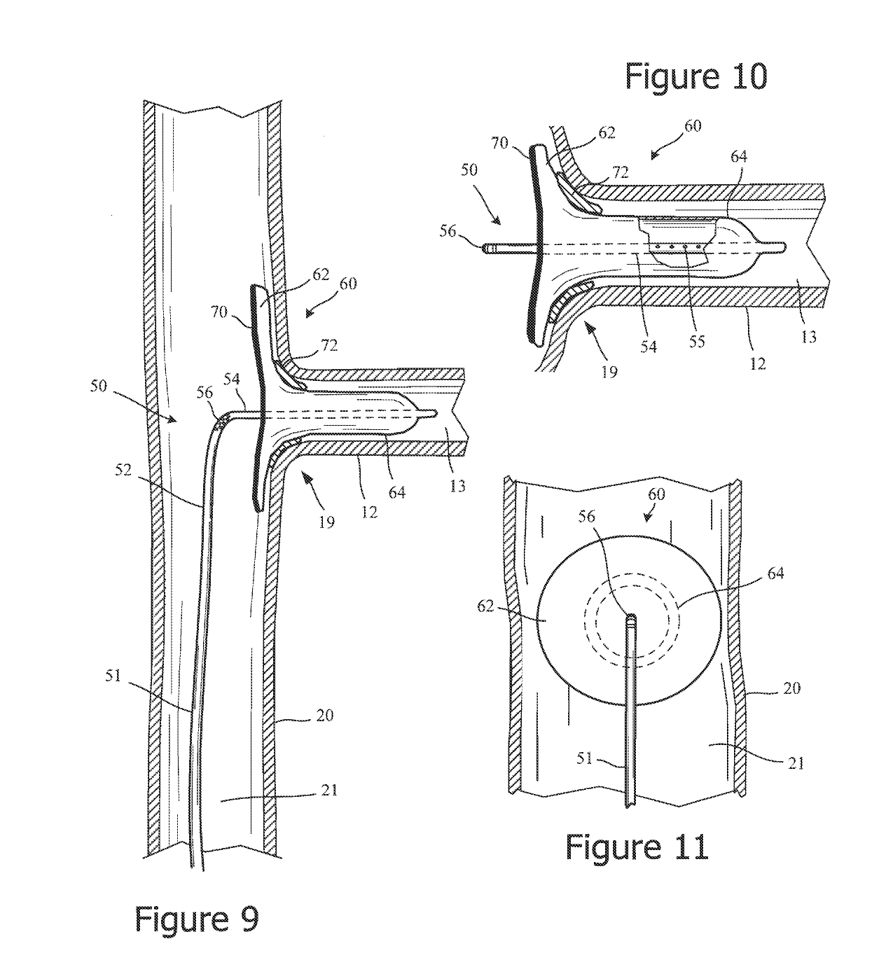

Turning now to FIGS. 9-11, there is illustrated various views of a cryoballoon catheter 50 implemented in accordance with embodiments of the present invention. The cryoballoon catheter 50 is shown in an inflated configuration deployed at the ostium 19 of a renal artery 12. FIG. 9 provides a sectional view of the cryoballoon catheter 50 deployed within aortal/renal vasculature, with FIG. 10 showing a partial cut-away of the cryoballoon 60 and FIG. 11 showing a rear view of the cryoballoon catheter 50 in a deployed state.

The cryoballoon 60 includes an ostial balloon 62 that has a flattened proximal section 70 relative to its distal treatment section. The flattened profile of the proximal section 70 serves to decrease the volume of the ostial balloon 62 within the lumen 21 of the aorta 20 when the cryoballoon catheter 50 is deployed and inflated at the ostium 19 of the renal artery 12, thereby reducing occlusion of the blood flowing through the aorta 20. The flattened profile of the proximal section 70 may be achieved by constructing this portion of the ostial balloon 62 with a wall thickness greater than that of the distal section, by use of a balloon construction material(s) of reduced elasticity relative to that used in the distal section, and/or by provision of thermal insulation that renders the proximal section 70 less resilient than the distal section of the ostial balloon 62.

An alignment element 72 is shown provided proximate a transition region between the ostial and arterial balloons 62, 64 of the cryoballoon 60. The alignment element 72 is preferably configured to facilitate proper positioning of the cryoballoon 60 at the renal artery during cryoballoon deployment. As was discussed previously, the alignment element 72 may be a feature integral to the cryoballoon 60 or a separate element that is bonded, welded or otherwise affixed at the transition region of the cryoballoon 60. The alignment element 72 may extend circumferentially around the transition region of the cryoballoon 60 or be situated at one or more discrete locations at or around the transition region of the cryoballoon 60. As was also discussed, the alignment element 72 is preferably formed of a thermally conductive material and/or has the property of moderating thermal conduction at the ostial treatment site. In some embodiments, the alignment element 72 is configured as a primary cryotherapy delivery component for cryogenically treating the ostium 72 of the renal artery 12, and may be constructed to facilitate flow of a cryogen therethrough.

In the cut-away portion of the cryoballoon 60 shown in FIG. 10, a distal section 54 of the catheter 51 includes a manifold arrangement 55 that includes various ports. The configuration of the manifold arrangement 55 varies in accordance with the construction particulars of the cryoballoon 62. For example, the manifold arrangement 55 may incorporate ports and possibly tubes that provide supply and exhaust/return conduits for one or multiple balloons. Some balloons may be constructed to receive and exhaust cryogenic fluid, while other are implemented to receive and return saline or similar pressurizing fluid. As was previously discussed, the arterial balloon 64 may be constructed to include cryogenic treatment elements, as is shown in the embodiment of FIG. 10, or may be implemented without cryogenic treatment elements and used primarily as a positioning or stabilizing balloon.

FIGS. 9-11 show a hinge mechanism 56 built into the cryoballoon catheter 50 proximate the cryoballoon 60. The hinge mechanism 56 is constructed to enhance user manipulation of the cryoballoon catheter 50 when navigating the cryoballoon catheter 50 around a nearly 90 degree turn from the abdominal aorta 20 into the ostium 19 of the renal artery 12. Integration of a hinge mechanism 56 into the cryoballoon catheter 50 advantageously reduces the force that the cryoballoon 60 may impart on the renal artery 12 during the freeze/thaw cycle.

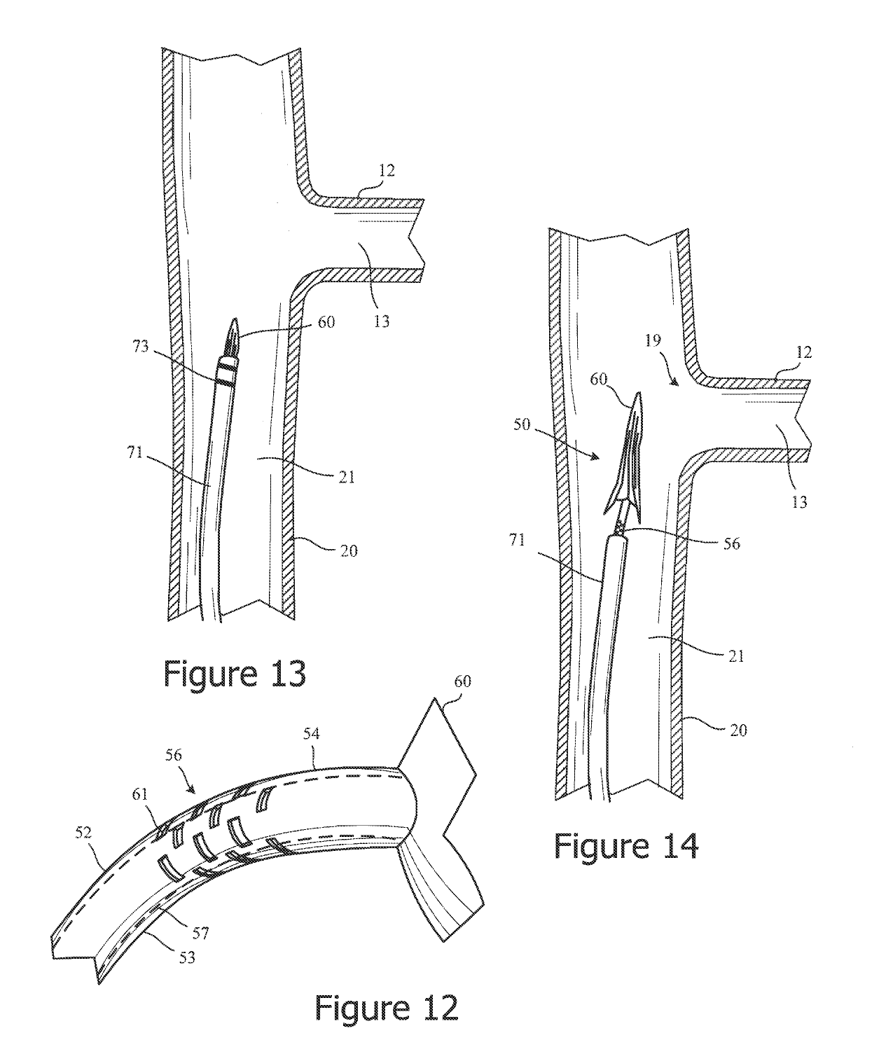

FIG. 12 illustrates a portion of the cryoballoon catheter 50 that incorporates a hinge mechanism 56 in accordance with embodiments of the invention. The hinge mechanism 56 is provided at a location of the catheter 51 between a proximal section 52 and a distal section 54 of the catheter 51. The hinge mechanism 56 is preferably situated near the proximal section of the cryoballoon 60. According to various embodiments, the hinge mechanism 56 comprises a slotted tube arrangement that is configured to provide a flexible hinge point of the catheter 51 proximate the cryoballoon 60.

The catheter 51 may be formed to include an elongate core member 57 and a tubular member 53 disposed about a portion of the core member 57. The tubular member 53 may have a plurality of slots 61 formed therein. The slotted hinge region of the catheter 51 may be configured to have a preferential bending direction.

For example, and as shown in FIG. 12, tubular member 52 may have a plurality of slots 61 that are formed by making a pair of cuts into the wall of tubular member 61 that originate from opposite sides of tubular member 53, producing a lattice region of greater flexibility relative to the proximal and distal sections 51, 54 of the catheter 51. The thickness of the catheter wall at the hinge region 56 can be varied so that one side of the catheter wall is thicker than the opposite side. This difference in wall thickness alone or in combination with a difference in slot (void) density at the hinge region 56 provides for a preferential bending direction of the distal portion of the cryoballoon catheter 50.

A hinge arrangement 56 constructed to provide for a preferential bending direction allows a physician to more easily and safely navigate the cryoballoon catheter 50 to make the near 90 degree turn into the renal artery from the abdominal aorta 20. One or more marker bands may be incorporated at the hinge region 56 to provide visualization of this region of the catheter 51 during deployment. Details of useful hinge arrangements that can be incorporated into embodiments of a cryoballoon catheter 50 of the present invention are disclosed in U.S. Patent Publication Nos. 2008/0021408 and 2009/0043372, which are incorporated herein by reference. It is noted that the cryoballoon catheter 50 may incorporate a steering mechanism in addition to, or exclusion of, a hinge arrangement 56. Known steering mechanisms incorporated into steerable guide catheters may be incorporated in various embodiments of a cryoballoon catheter 50 of the present invention.

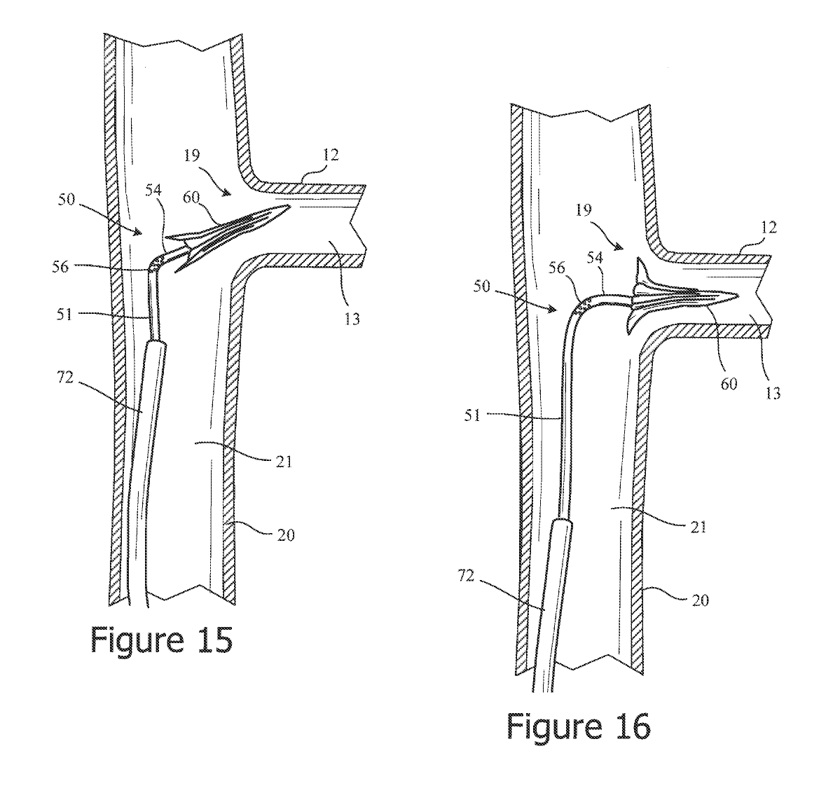

FIGS. 13-16 illustrate a series of views of a cryoballoon catheter 50 of the present invention at different states of deployment within a patient. A typical deployment procedure involves percutaneous delivery of a guide catheter 71 to an access vessel, via an introducer sheath (not shown), and advancement of the guide catheter 71 through access vasculature to the abdominal aorta at a location superior or inferior to the renal artery 12. The guide catheter 71 preferably includes one or more marker bands 73 to aid in visualization of at least the distal open tip of the guide catheter 71. The guide catheter 71 may include a steering mechanism, of a type discussed above.

With the guide catheter 71 positioned near the ostium 19 of the renal artery 12, the cryoballoon catheter 50, in a collapsed configuration, is advanced through the lumen of the guide catheter 71. Marker bands provided on the arterial and ostial balloons 64, 62 of the cryoballoon 60 facilitates visualization of the cryoballoon catheter 50 when advancing the cryoballoon catheter 50 through the guide catheter 71. As is shown in FIG. 16, the cryoballoon catheter 50 is advanced out of the guide catheter 71, allowing the cryoballoon 60 to expand somewhat upon exiting the distal open tip of the guide catheter 71. As the region of the catheter 51 comprising the hinge mechanism 56 passes out of the guide catheter 71, the distal portion 54 of the catheter 51 preferably bends relative to the proximal portion 52 of the catheter 51 in a direction dictated by the preferential bend provided by the hinge mechanism 56. The catheter 51 may be rotated by the physician to achieve proper orientation of the cryoballoon 60 relative to the ostium 19 of the renal artery 12.

Further advancement of the cryoballoon catheter 50 (or retraction of the guide catheter 71) relative to the guide catheter 71 allows for an increase in bend angle at the hinge region 56, allowing the physician to safely advance the distal tip of the cryoballoon 60 into the ostium 19 of the renal artery lumen 13. As was discussed previously, the cryoballoon 60 may be slightly pressurized with saline or similar fluid to help seat the ostial balloon 62 against the ostium 19 of the renal artery 12. Pressurization of the arterial balloon 64 may also aid in cannulating the cryoballoon catheter 50 within the renal artery 12. The ostial balloon section 62 of the cryoballoon catheter 50 is preferably seated against the ostium 19, at which point cryogenic therapy may be initiated by the physician.

Embodiments of the present invention may be implemented to provide varying degrees of cryotherapy to the ostium 19 and other innervated renal vasculature. For example, embodiments provide for control of the extent and relative permanency of renal nerve impulse transmission interruption achieved by cryotherapy delivered using a cryoballoon catheter 50 of the present invention. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Renal nerve fiber regeneration and re-innervation may be permanently compromised by applying cryogenic therapy to innervated renal vasculature, including the ostium 19 and renal ganglia, at a sufficiently low temperature to allow ice crystals to form inside nerve fibers 14b. Formation of ice crystals inside nerve fibers 14b of innervated renal arterial tissue and renal ganglia tears the nerve cells apart, and physically disrupts or separates the endoneurium tube, which can prevent regeneration and re-innervation processes. Delivery of cryogenic therapy to renal nerves 14 at a sufficiently low temperature in accordance with embodiments of the present invention can cause necrosis of renal nerve fibers 14b, resulting in a permanent and irreversible loss of the conductive function of renal nerve fibers 14b.

With continued reference to FIGS. 3B and 3C, major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation. The degree of interruption of action potential transmission along nerve fibers 14b of innervated renal arterial tissue and renal ganglia may be varied by delivering cryogenic therapy to aortal/renal vasculature having different temperature and duration parameters.

In some embodiments, a cryoballoon catheter 50 of the present invention may be implemented to deliver a cryotherapy that causes transient and reversible injury to renal nerve fibers 14b. In other embodiments, a cryoballoon catheter 50 of the present invention may be implemented to deliver a cryotherapy that causes more severe injury to renal nerve fibers 14b, which may be reversible if cryotherapy is terminated in a timely manner. In preferred embodiments, a cryoballoon catheter 50 of the present invention may be implemented to deliver a cryotherapy that causes severe and irreversible injury to renal nerve fibers 14b, resulting in permanent cessation of renal sympathetic nerve activity. For example, a cryoballoon catheter 50 may be implemented to deliver a cryotherapy that causes formation of ice crystals sufficient to physically separate the endoneurium tube of the nerve fiber 14b, which can prevent regeneration and re-innervation processes.

By way of example, and in accordance with Seddon's classification as is known in the art, a cryoballoon catheter 50 may be implemented to deliver a cryotherapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a cryoballoon catheter 50 of the present invention.

A cryoballoon catheter 50 may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A cryoballoon catheter 50 may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

In some embodiments, cryotherapy delivered by a cryoballoon catheter 50 of the present invention may be controlled to achieve a desired degree of attenuation in renal nerve activity. Selecting or controlling cryotherapy delivered by the cryoballoon catheter 50 advantageously facilitates experimentation and titration of a desired degree and permanency of renal sympathetic nerve activity cessation.

In general, embodiments of a cryoballoon catheter 50 may be implemented to deliver cryogenic therapy to cause renal denervation at therapeutic temperatures ranging between approximately 0° C. and approximately −180° C. For example, embodiments of a cryoballoon catheter 50 may be implemented to deliver cryogenic therapy to cause renal denervation with temperatures at the renal nerves ranging from approximately 0° C. to approximately −30° C. at the higher end, and to about −140° C. to −180° C. at the lower end. Less robust renal nerve damage is likely for temperatures approaching and greater than 0° C., and more robust acute renal denervation is likely for temperatures approaching and less than −30° C., for example, down to −120 C. to −180 C. These therapeutic temperature ranges may be determined empirically for a patient, a patient population, or by use of human or other mammalian studies.

It has been found that delivering cryotherapy to the ostium of the renal artery and to the renal ganglia at a sufficiently low temperature with freeze/thaw cycling allows ice crystals to form inside nerve fibers 14b and disrupt renal nerve function and morphology. For example, achieving therapeutic temperatures that range from −30° C. to +10° C. at a renal nerve for treatment times of 30 seconds to 4 minutes and thaw times of about 1 to 2 minutes has been found to cause acute renal denervation in at least some of the renal nerves in a porcine model.

The representative embodiments described below are directed to cryoballoon catheters of the present invention configured for delivering cryogenic therapy to renal vasculature at specified therapeutic temperatures or temperature ranges, causing varying degrees of nerve fiber degradation. As was discussed above, therapeutic temperature ranges achieved by cryoballoon catheters of the present invention may be determined using non-human mammalian studies. The therapeutic temperatures and degrees of induced renal nerve damage described in the context of the following embodiments are based largely on cryoanalgesia studies performed on rabbits (see, e.g., L. Zhou et al., Mechanism Research of Cryoanalgesia, Neurological Research, Vol. 17, pp. 307-311 (1995)), but may generally be applicable for human renal vasculature. As is discussed below, the therapeutic temperatures and degrees of induced renal nerve damage may vary somewhat or significantly from those described in the context of the following embodiments based on a number of factors, including the design of the cryotherapy apparatus, duration of cryotherapy, and the magnitude of mechanical disruption of nerve fiber structure that can be achieved by subjecting renal nerves to freeze/thaw cycling, among others.

In accordance with various embodiments, a cryoballoon catheter 50 of the present invention may be implemented to deliver cryogenic therapy to cause a minimum level of renal nerve damage. Cooling renal nerve fibers to a therapeutic temperature ranging between about 0° C. and about −20° C. is believed sufficient to temporarily block some or all renal sympathetic nerve activity and cause a minimum degree of renal nerve damage, consistent with neurapraxia for example. Freezing renal nerves to a therapeutic temperature of −20° C. or higher may not cause a permanent change in renal nerve function or morphology. At therapeutic temperatures of −20° C. or higher, slight edema and myelin swelling may occur in some of the renal nerve fibers, but these conditions may be resolved after thawing.

In other embodiments, cooling renal nerve fibers to a therapeutic temperature ranging between about −20° C. and about −60° C. is believed sufficient to block all renal sympathetic nerve activity and cause an intermediate degree of renal nerve damage, consistent with axonotmesis (and possibly some degree of neurotmesis for lower temperatures of the −20° C. and −60° C. range), for example. Cooling renal nerves to a therapeutic temperature of −60° C. may cause freezing degeneration and loss of renal nerve conductive function, but may not result in a permanent change in renal nerve function or morphology. However, renal nerve regeneration is substantially slowed (e.g., on the order of 90 days). At a therapeutic temperature of −60° C., the frozen renal nerve is likely to demonstrate edema with thickening and loosening of the myelin sheaths and irregular swelling of axons, with Schwann cells likely remaining intact.

In further embodiments, cooling renal nerve fibers to a therapeutic temperature ranging between about −60° C. and about −100° C. is believed sufficient to block all renal sympathetic nerve activity and cause an intermediate to a high degree of renal nerve damage, consistent with neurotmesis, for example. Cooling renal nerves to a therapeutic temperature of −100° C., for example, causes swelling, thickening, and distortion in a large percentage of axons. Exposing renal nerves to a therapeutic temperature of −100° C. likely causes splitting or focal necrosis of myelin sheaths, and microfilament, microtubular, and mitochondrial edema. However, at a therapeutic temperature of −100° C., degenerated renal nerves may retain their basal membranes, allowing for complete recovery over time.

Although substantially slowed (e.g., on the order of 180 days), renal nerve regeneration may occur and be complete.

In accordance with other embodiments, cooling renal nerve fibers to a therapeutic temperature of between about −140° C. and about −180° C. is believed sufficient to block all renal sympathetic nerve activity and cause a high degree of renal nerve damage, consistent with neurotmesis for example. Application of therapeutic temperatures ranging between about −140° C. and about −180° C. to renal nerve fibers causes immediate necrosis, with destruction of basal membranes (resulting in loss of basal laminea scaffolding needed for complete regeneration). At these low temperatures, axoplasmic splitting, axoplasmic necrosis, and myelin sheath disruption and distortion is likely to occur in most renal nerve fibers. Proliferation of collagen fibers is also likely to occur, which restricts renal nerve regeneration.

It is believed that exposing renal nerves to a therapeutic temperature of about −140° C. or lower causes permanent, irreversible damage to the renal nerve fibers, thereby causing permanent and irreversible termination of renal sympathetic nerve activity. For some patients, exposing renal nerves to a therapeutic temperature ranging between about −120° C. and about −140° C. may be sufficient to provide similar permanent and irreversible damage to the renal nerve fibers, thereby causing permanent and irreversible cessation of renal sympathetic nerve activity. In other patients, it may be sufficient to expose renal nerves to a therapeutic temperature of at least −30° C. in order to provide a desired degree of renal sympathetic nerve activity cessation.

In preferred embodiments, it is desirable that the cryogen used to deliver cryotherapy to renal vasculature be capable of freezing target tissue so that nerve fibers innervating the ostium 19 and renal artery 12 are irreversibly injured, such that nerve conduction along the treated renal nerve fibers is permanently terminated. Suitable cryogens include those capable of cooling renal nerve fibers and renal ganglia to temperatures of at least about −120° C. or lower, preferably to temperatures of at least about −130° C. or lower, and more preferably to temperatures of at least about −140° C. or lower. It is understood that use of cryogens that provide for cooling of renal nerve fibers and renal ganglia to temperatures of at least about −30° C. may effect termination of renal sympathetic nerve activity with varying degrees of permanency.

The temperature ranges and associated degrees of induced renal nerve damage described herein are provided for non-limiting illustrative purposes. Actual therapeutic temperatures and magnitudes of resulting nerve injury may vary significantly from those described herein, and be impacted by a number of factors, including patient-specific factors (e.g., the patient's unique renal vasculature and sympathetic nervous system characteristics), therapy duration, frequency and duration of freeze/thaw cycling, structural characteristics of the cryotherapy balloon arrangement, type of cryogen used, and method of delivering cryotherapy, among others.

It is believed that higher degrees of renal nerve injury may be achieved by subjecting renal nerves to both cryotherapy and freeze/thaw cycling when compared to delivering cryotherapy without employing freeze/thaw cycling. Implementing freeze/thaw cycling as part of cryotherapy delivery to renal nerves may result in achieving a desired degree of renal sympathetic nerve activity attenuation (e.g., termination) and permanency (e.g., irreversible) at therapeutic temperatures higher than those discussed above. Various thermal cycling parameters may be selected for, or modified during, renal denervation cryotherapy to achieve a desired level of renal nerve damage, such parameters including the number of freeze/thaw cycles, high and low temperature limits for a given freeze/thaw cycle, the rate of temperature change for a given freeze/thaw cycle, and the duration of a given freeze/thaw cycle, for example. As was previously discussed, these therapeutic temperature ranges and associated degrees of induced renal nerve damage may be determined empirically for a particular patient or population of patients, or by use of human or other mammalian studies.

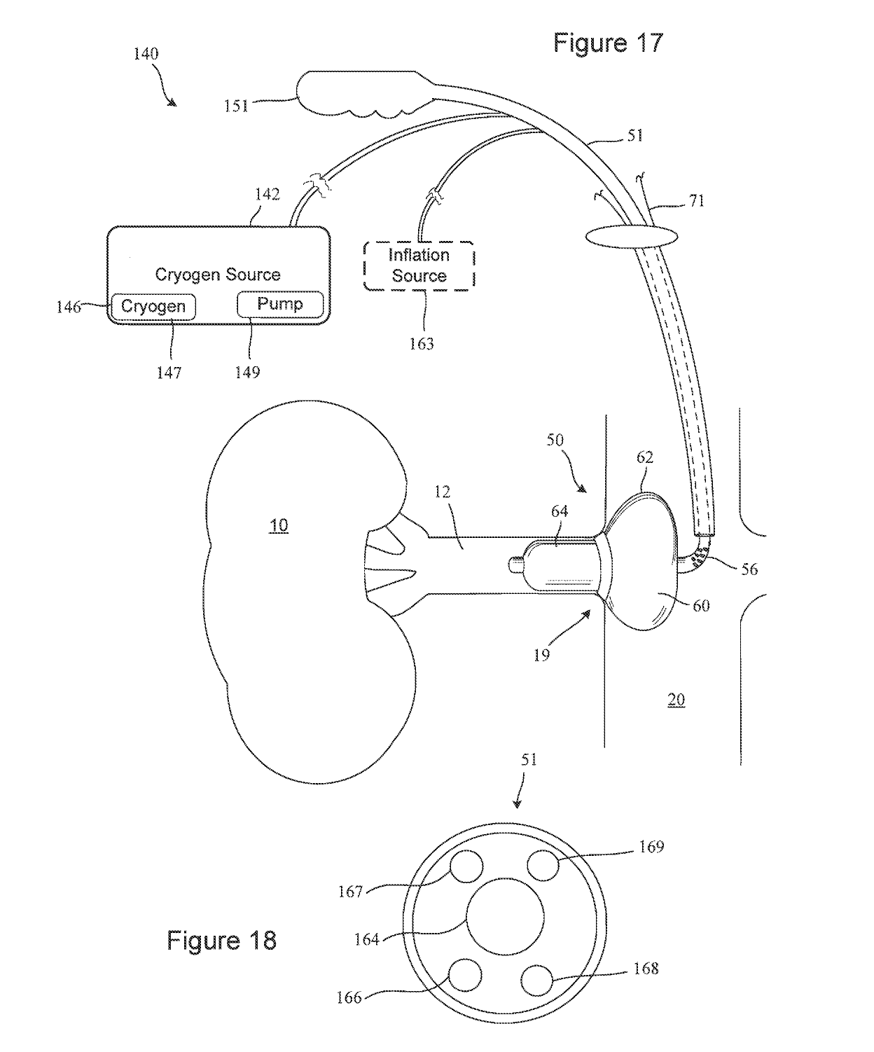

FIG. 17 shows a medical system 140 configured to facilitate intravascular access to the renal artery 12 and deliver cryogenic denervation therapy to renal nerves and ganglia at an ostial region of the renal artery 12 that contribute to renal sympathetic nerve activity in accordance with embodiments of the present invention. A cryogen source 142 includes a reservoir 147 fluidly coupled to a pump 149. A cryogen 146 is contained within the reservoir 147. Achieving desired therapeutic temperatures at targeted renal nerve fibers is largely dictated by the thermal transfer properties of the selected cryogen and design of the cryotherapy balloon catheter 50. A variety of useful cryogens 146 may be employed, including saline, a mixture of saline and ethanol, Freon or other fluorocarbons, nitrous oxide, liquid nitrogen, and liquid carbon dioxide, for example.

As is illustrated in FIG. 17, the cryogen source 142 is fluidly coupled to a cryoballoon catheter 50. The catheter 51 is preferably lined with or otherwise incorporates insulation material(s) having appropriate thermal and mechanical characteristics suitable for a selected cryogen. A lumen arrangement is shown in FIG. 18 that can include a number of lumens depending on the particular implementation of the cryoballoon catheter 50. The lumen arrangement of FIG. 18 is shown for illustrative purposes only, and is not intended to limit the configuration and/or functionality of the cryoballoon catheter 50. Accordingly, particular lumens shown in FIG. 18 need not be incorporated in a given cryoballoon catheter 50. Alternatively, lumens other than those shown in FIG. 18 may be incorporated in a given cryoballoon catheter 50, including lumens formed on the exterior wall of the catheter's shaft.

In some embodiments, the lumen arrangement includes a first lumen 166, for supplying a cryogen to the distal end of the catheter 51, and a second lumen 168, for returning the cryogen or exhaust gas to the proximal end of the catheter 51. The supply and return lumens 166, 168 are fluidly coupled to a cryoballoon 60 disposed at the distal end of the catheter 51. The cryogen may be circulated through the cryoballoon 60 via a hydraulic circuit that includes the cryogen source 142, supply and return lumens 166, 168, and the cryoballoon 60 disposed at the distal end of the catheter 51.

The supply lumen 166 may be supplied with a pressurized cryogen by the cryogen source 142 that both pressurizes the cryoballoon 60 and provides the cryogen to the cryoballoon 60. In some configurations, the catheter 51 may include one or more inflation lumens (e.g., lumens 167 and/or 169) that fluidly communicate with one or more dilation or stabilizing balloons disposed at the distal end of the catheter 51. In further embodiments, one or more cryoballoons and one or more dilation/stabilizing balloons may be incorporated at the distal end of the catheter 51, with appropriate supply, return, and pressurization lumens provided to fluidly communicate with the cryogen source 142 and an optional inflation fluid (e.g., saline) source 163. The catheter 51 may optionally include a main lumen 164 configured to receive a guide wire for embodiments that employ an over-the-wire deployment approach.

Embodiments of the present invention may incorporate selected balloon, catheter, lumen, control, and other features of the devices disclosed in the following commonly owned U.S. patents and published patent applications: U.S. Patent Publication Nos. 2009/0299356, 2009/0299355, 2009/0287202, 2009/0281533, 2009/0209951, 2009/0209949, 2009/0171333, 2009/0171333, 2008/0312644, 2008/0208182, 2008/0058791 and 2005/0197668, and U.S. Pat. Nos. 5,868,735, 6,290,696, 6,648,878, 6,666,858, 6,709,431, 6,929,639, 6,989,009, 7,022,120, 7,101,368, 7,172,589, 7,189,227, and 7,220,257, which are incorporated herein by reference. Embodiments of the present invention may incorporate selected balloon, catheter, and other features of the devices disclosed in U.S. Pat. Nos. 6,355,029, 6,428,534, 6,432,102, 6,468,297, 6,514,245, 6,602,246, 6,648,879, 6,786,900, 6,786,901, 6,811,550, 6,908,462, 6,972,015, and 7,081,112, which are incorporated herein by reference.

The catheter apparatus shown in FIGS. 17 and 18 may incorporate a proximal section that includes a control mechanism 151 to facilitate physician manipulation of the catheter apparatus 50. In certain embodiments, the control mechanism 151 facilitates physician manipulation of the guide catheter 71 and the cryoballoon catheter 50, such as delivery and deployment of the guide catheter 71 and cryoballoon catheter 50 to the renal artery 12. In some configurations, the control mechanism 151 may include a steerable portion that facilitates physician control of rotation and longitudinal displacement of one or both of the guide catheter 71 and cryoballoon catheter 50 through the access vasculature and into the renal artery 12. The control mechanism 151 may accommodate a number of physician tools that are external of a patient's body when in use, and allow the physician to perform various functions at the distal section of the catheter apparatus. Each of the tools may be coupled to one or more associated lumens in the catheter apparatus using one or more manifolds at the proximal section, for example.

The following is a representative example of a cryotherapy procedure that employs a cryoballoon catheter 50 for denervating the ostium of the renal artery and, optionally, other innervated renal vasculature in accordance with embodiments of the present invention. During a first stage of the representative cryotherapy procedure, the cryoballoon catheter 50 is advanced to an aortal location proximate the ostium 19 of the renal artery 12, preferably as described previously with reference to FIGS. 13-15. With the cryoballoon 60 positioned at the ostium 19, the balloon arrangement is partially inflated, preferably with saline but alternatively with a cryogenic fluid delivered and exhausted at a low flow rate. The flow rate of the saline or cryogenic fluid can be controlled by the inflation source 163 and/or cryogen source 142 such that a pressure inside the ostial balloon 62 is developed that is sufficient to push the outer surface of the ostial balloon 62 against tissue of the ostium 19 of the renal artery 12.

During a second stage of this representative example, an increased volume of cryogenic fluid can be supplied to the ostial balloon 62 in order to cool the treatment surface of the ostial balloon 62 via the Joule-Thomson effect. Cryogenic fluid may also be delivered to the arterial balloon 64 in order to cool the treatment surface of the arterial balloon 64. Alternatively, the arterial balloon 64 may be pressurized with saline or similar fluid, as discussed previously. During the second stage, the flow rate of cryogenic fluid through the cryoballoon 60 is regulated at a desired therapeutic rate, by which heat is extracted from the tissue surrounding the treatment region at a rate sufficient to cool a desired amount of ostial tissue to a therapeutically low temperature, such as a temperature between 0° C. to −180° C.

By controlling both the rate at which cryogenic fluid is delivered to the cryoballoon 60 and the rate at which exhaust gas or liquid is extracted from the cryoballoon 60, the cryogen source controller can develop and maintain a pressure inside the cryoballoon 60 at a number of different temperatures. Other useful devices and methodologies that may be implemented by a medical system 140 for controlling a cryotherapy delivered by a cryoballoon catheter 60 of the present invention are disclosed in commonly owned U.S. Published Patent No. 2009/0299356 and 2005/0197668, which are incorporated herein by reference.

Embodiments of a cryoballoon of the present invention may be implemented to incorporate features in addition to, or different from, those described hereinabove. For example, a cryoballoon may incorporate ribs, flutes, and other structural features that serve to facilitate preferential balloon expansion. Such ribbed and fluted structures may be formed by varying balloon wall thicknesses and/or incorporating different balloon materials at selected balloon locations. Ribs, flutes, and/or diversion channels or conduits may be incorporated into a cryoballoon for purposes of providing or increasing blood perfusion through or around the cryoballoon, particularly when the cryoballoon is inflated within the abdominal aorta and renal artery. Tissues in contact with flowing blood may be protected from thermal damage.

Non-uniformity of cryoballoon geometry may be achieved in various ways, including those discussed hereinabove. In some embodiments, a cryoballoon of the present invention may include an ostial balloon section having a greater circumferential surface area than an arterial balloon section. In other embodiments, the arterial balloon section may have a greater longitudinal circumferential surface area than the ostial balloon section. Embodiments of a cryoballoon of the present invention may have a generally triangular longitudinal cross-section, a generally T-shaped longitudinal cross-section, or a generally dog leg-shaped longitudinal cross-section, for example.

The foregoing description of the various embodiments of the invention has been presented for the purposes of illustration and description. It is not intended to be exhaustive or to limit the invention to the precise form disclosed. Many modifications and variations are possible in light of the above teaching. For example, the devices and techniques disclosed herein may be employed in vasculature of the body other than renal vasculature, such as coronary and peripheral vessels and structures. It is intended that the scope of the invention be limited not by this detailed description, but rather by the claims appended hereto.

What is claimed is:

1. An ablation balloon catheter apparatus, comprising:
a flexible shaft comprising a proximal end, a distal end, and a lumen arrangement extending between the proximal and distal ends, the shaft having a length sufficient to access a patient's renal artery relative to a percutaneous access location;
a balloon provided at the distal end of the shaft and fluidly coupled to the lumen arrangement, the balloon having a longitudinal axis extending from a proximal end to a distal end thereof; and
an ablation member extending helically around the balloon in an axial direction.

2. The apparatus of claim 1, wherein the ablation member has a first free end adjacent the proximal end of the balloon and a second free end adjacent the distal end of the balloon, wherein the ablation member is configured to create a non-continuous helical treatment pattern.

3. The apparatus of claim 1, wherein the ablation member is a separate element attached to the balloon.

4. The apparatus of claim 1, wherein the ablation member is a cryotherapy element.

5. The apparatus of claim 1, wherein the balloon comprises a distal section dimensioned for placement within a renal artery and a proximal section dimensioned to abut against an ostium of the renal artery and extend into at least a portion of the patient's abdominal aorta, wherein a diameter of the proximal section is greater than a diameter of the distal section.

6. The apparatus of claim 5, wherein the diameter of the proximal section is at least 200% greater than the diameter of the distal section.

7. The apparatus of claim 5, wherein the distal and proximal sections of the balloon are in fluid communication with each other such that both the proximal and distal sections inflate in response to receiving pressurized fluid and to deflate in response to removal of the fluid.

8. The apparatus of claim 5, wherein the ablation member is a cryotherapy element, wherein an outer surface of the distal section of the balloon is formed from a thermally insulating material and the cryotherapy element is formed from a conductive material disposed on the outer surface of the distal section of the balloon.

9. The apparatus of claim 5, wherein both the proximal and distal balloon sections are compliant.

10. The apparatus of claim 5, wherein the proximal balloon section is compliant and the distal balloon section is non-compliant.

11. The apparatus of claim 5, wherein at least the proximal balloon section includes an inner balloon disposed within an outer balloon.

12. The apparatus of claim 11, wherein the inner balloon and outer balloon are configured to receive different inflation fluids.

13. The apparatus of claim 5, further comprising a thermally conductive alignment element disposed on an outer surface of the balloon at a transition region between the proximal and distal balloon sections.

14. The apparatus of claim 1, further comprising a hinge mechanism provided on the flexible shaft proximal of the balloon, the hinge mechanism configured to facilitate preferential bending at the distal end to aid in directing the balloon into the renal artery from the abdominal aorta.

15. The apparatus of claim 5, wherein the distal section is cylindrical and has an expanded configuration in which a longitudinal dimension thereof is up to about 400% greater than a non-expanded length thereof while a diameter of the distal balloon section in the expanded configuration is about 0% to about 20% greater than its non-expanded diameter.

16. The apparatus of claim 5, wherein the ablation member is a cryotherapy element, wherein the balloon further comprises one or more thermal insulation layers disposed at a proximal portion of the proximal balloon section, the one or more thermal insulation layers providing thermal insulation between cryogenic fluid within the balloon and blood contacting the balloon catheter apparatus.

17. The apparatus of claim 5, wherein the distal section comprises a first material and the proximal section comprises a second material different from the first material.

18. The apparatus of claim 5, wherein the balloon comprises a wall, wherein the wall in the distal section has a first thickness and the wall in the proximal section has a second thickness different from the first thickness.

19. The apparatus of claim 5, wherein the distal section inflates more easily than the proximal section such that when the balloon receives pressurized inflation fluid, the distal section inflates before the proximal section.

20. A cryotherapy balloon catheter apparatus, comprising:
a flexible shaft comprising a proximal end, a distal end, and a lumen arrangement extending between the proximal and distal ends, the shaft having a length sufficient to access a patient's renal artery relative to a percutaneous access location;
a balloon provided at the distal end of the shaft and fluidly coupled to the lumen arrangement, the balloon comprising a cylindrical distal section dimensioned for placement within a renal artery and a proximal section dimensioned to abut against an ostium of the renal artery and extend into at least a portion of the patient's abdominal aorta, wherein the distal and proximal sections of the balloon are in fluid communication with each other such that both the proximal and distal sections inflate in response to receiving pressurized inflation fluid and to deflate in response to removal of the inflation fluid; and
a cryotherapy element extending helically around the cylindrical distal section of the balloon, the cryotherapy element having a first free end adjacent a proximal end of the distal section of the balloon and a second free end adjacent a distal end of the distal section of the balloon, wherein the cryotherapy element is configured to create a non-continuous helical treatment pattern.
